(19) United States
(12) Patent Application Publication
(10) Pub. No.: US 2018/0250167 A1
(43) Pub. Date: Sep. 6, 2018

(54) TOOL FOR CORNEAL AND SCLERAL INCISIONS

(71) Applicant: INDYWIDUALNA SPECJALISTYCZNA PRAKTYKA LEKARSKA DR MED. BARTLOMIEJ KALUZNY, Bydgoszcz (PL)

(72) Inventor: Agnieszka KALUZNA, Bydgoszcz (PL)

(21) Appl. No.: 15/756,319

(22) PCT Filed: Sep. 5, 2015
(86) PCT No.: PCT/PL2015/000142

§ 371 Date: Feb. 28, 2018

(51) Int. Cl. A61F 9/013; A61F 9/007

(52) U.S. Cl. CPC A61F 9/0133; A61F 9/00736

(57) ABSTRACT

Disclosed herein is a method and a tool for making tunnel incisions of modified architecture in the cornea and sclera. The tool comprises means for making two additional backward incisions made near the side edges of the main tunnel incision. Additional incisions, made to a depth of 10 to 300 μm, are set at an angle of from 15 to 165° relative to the plane of the main tunnel incision. Performing the additional incisions results in a partial downward incision of the tunnel floor (rear part) in its side.

### FIELD OF THE INVENTION

The present invention relates to a method and tool for self-sealing corneal and scleral valve incisions.

### BACKGROUND

The wall of the eyeball consists of three layers: a fibrous membrane (tunica fibrosa bulbi), uveitis (tunica vasculosa bulbi) and the inner membrane of the eyeball (tunica interna bulbi). The outermost layer, the fibrous membrane, provides the shape and biomechanical resistance of the eye, protects the tissue lying inside, and is essential for maintaining a positive pressure inside the eye. The fibrous membrane of the eye consists of the cornea, which is the front one sixth and sclera constituting the rear five-sixths. The cornea is the front, transparent wall portion of the eyeball distinguished by greater curvature. Corneal thickness at the center is generally within the range of 500-600 μm, and increases in thickness toward the periphery to 600-800 μm. The thickness of the sclera in the front part of the eyeball also exceeds 500 μm, with the exception of places where oculomotor muscles have their insertions.

The appropriate thickness and stiffness of the fibrous membrane are of great importance in ophthalmic microsurgery. They make tunnel incisions possible to perform, which enables the wound closure without the need for sutures. These types of incisions have been used for many years, particularly in cataract surgery using diamond or metal knives. In order to obtain tightness in most cases, however, they require administration of fluid into the stroma of the cornea in the incision area. This results in local tissue edema and leads to closure of the wound. Tunnel incisions are most often performed during the cataract phacoemulsification. In these cases, the tunnel incision performed in the corneal stroma is 0.8 to 4.0 mm wide to 0.8 mm wide and 1.5 to 2.5 mm long. Each tunnel incision has an entrance on the outer surface of the cornea and an exit on the inner surface. Immediately after performing the cut, the incision wound input and output have a linear shape. The incisions may be performed in a single plane or have a more complex shape of a tunnel—multifaceted. This architecture is designed to prevent the cutting movement of the substrate tunnel (rear part) with respect to the ceiling (front part) and provide a better sealing of the wound. After inserting the microsurgical tool into the incision area, i.e. usually the tip of a cataract phacoemulsification system, the edges of the tunnel get stretched, and the input and output wounds change their linear shape into to an oval one. These disorders in the shaping of the incision tunnel reduce the tendency of the incision to self-seal and require a significant amount of intracorneal fluid to prompt the swelling of the stroma around the wound and its closure. Such a procedure extends the healing time, the swelling may remain even for a few days. Moreover, it is not always possible to obtain a tight wound without sutures.

In recent years, the development of laser technology, in particular the development of the femtosecond laser for corneal surgery, made it possible to perform tunnel incisions in the cornea by means of a laser. The architecture of the incisions made in the transparent cornea is similar to those made with a knife. The repeatability of the incision shape is much greater. However, it is still necessary to administer fluid into the stroma of the cornea and the difficulty of obtaining tightness still remains in certain cases.

### SUMMARY

The invention relates to a method and a tool for making tunnel incisions of modified architecture in the cornea and sclera. The tool comprises means for making two additional backward incisions made near the side edges of the main tunnel incision. Additional incisions, made to a depth of 10 to 300 μm, are set at an angle of from 15 to 165° relative to the plane of the main tunnel incision. Performing the additional incisions results in a partial downward incision of the tunnel floor (rear part) in its side parts. In the area of the initial wound at the posterior corneal surface the additional incisions cause the inner layers of the corneal stroma and Descemet's membrane to form a kind of flap. Under the influence of the intraocular pressure the flap is pressed against the front part of the tunnel facilitating closure of the initial wound. The flap forms a kind of valve and facilitates the sealing of the wound.

### BRIEF DESCRIPTION OF THE DRAWINGS

FIG. 1 depicts a view of the tool according to a preferred embodiment of the present invention.

FIG. 2 depicts an enhanced bottom perspective view of the cutting tip of the tool according to FIG. 1.

FIG. 3 depicts a top perspective view of the cutting tip according to FIG. 2.

FIG. 4 depicts a front view of the cutting tip according to FIG. 2.

FIG. 5 depicts a rear view of the cutting tip according to FIG. 2.

FIG. 6 depicts a right side view of the cutting tip according to FIG. 2.

FIG. 7 depicts a left side view of the cutting tip according to FIG. 2.

FIG. 8 depicts a top view of the cutting tip according to FIG. 2.

FIG. 9 depicts a bottom view of the cutting tip according to FIG. 2.

FIGS. 10 and 11 depict variants of shapes of tunnel that can be made using the tool of the present invention.

FIGS. 12-14 depict views of the tool according to an alternate embodiment of the present invention.

FIGS. 15-17 depict views of the tool according to an alternate embodiment of the present invention.

FIGS. 18-20 depict views of the tool according to an alternate embodiment of the present invention.

### DETAILED DESCRIPTION

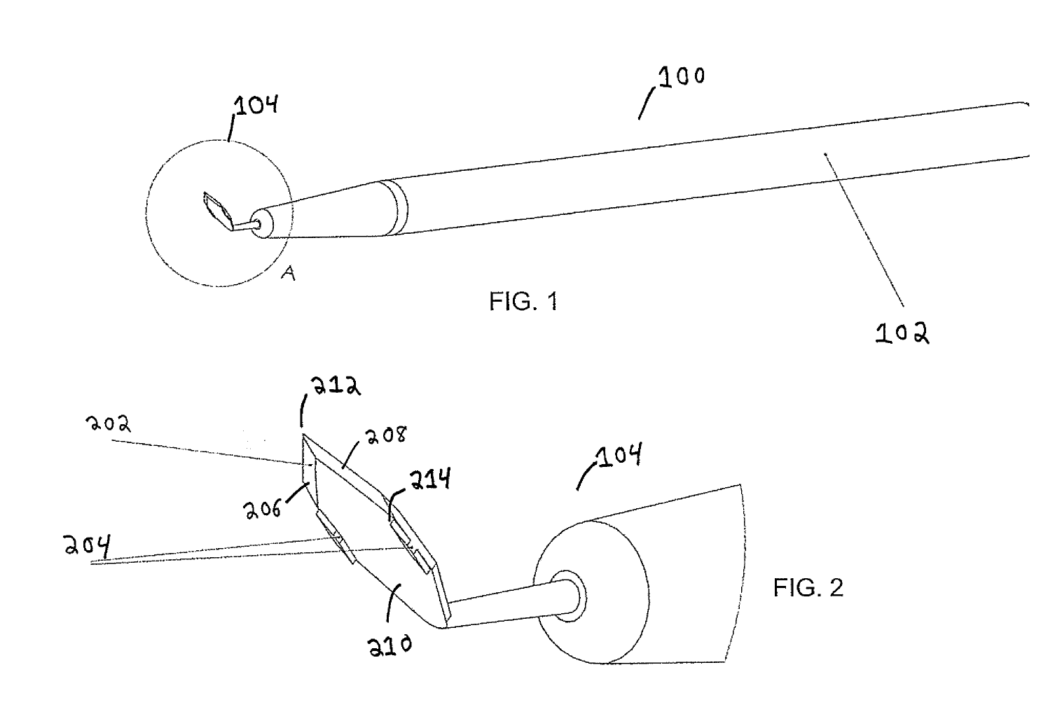

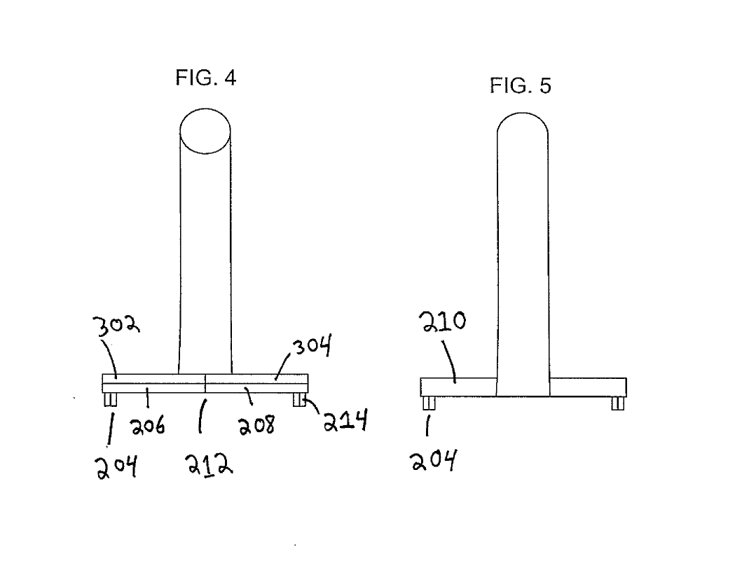

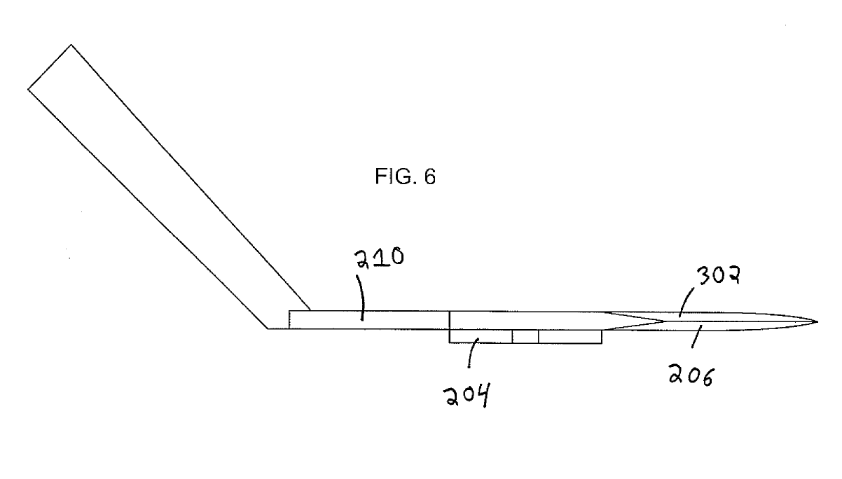

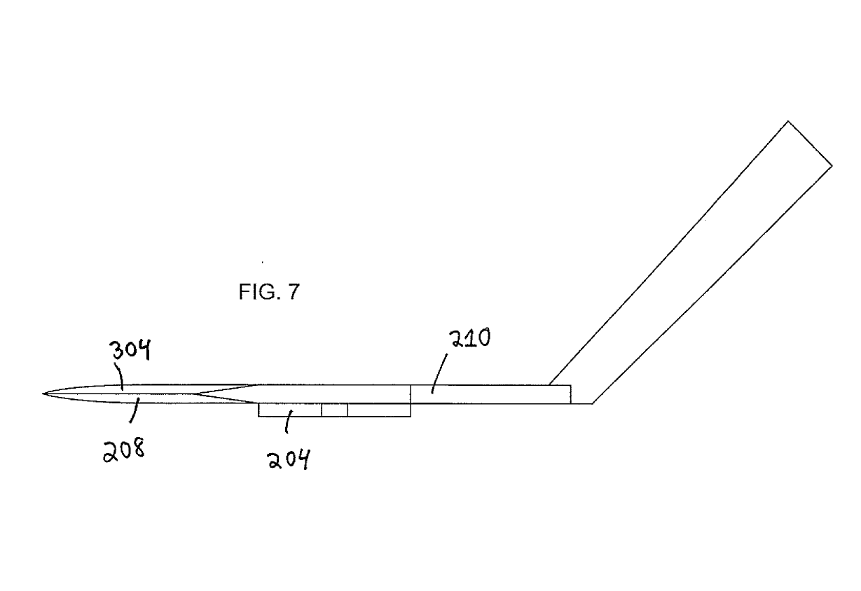

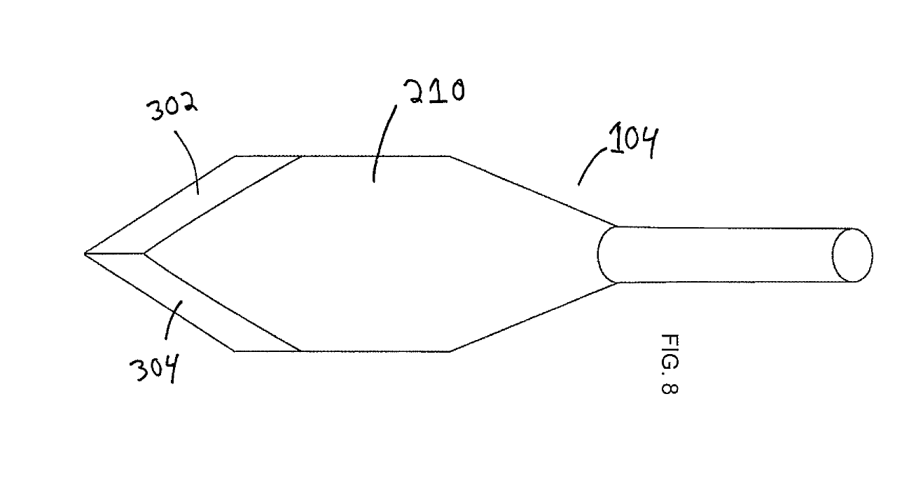

With reference to FIG. 1, depicted is a tool 100 for performing corneal and scleral incisions in the eye. Tool 100 generally comprises handle 102 to be operated by a surgeon and cutting tip 104. Cutting tip 104 is shown in greater detail in FIGS. 2-9. First, with reference to FIG. 2, cutting tip 104 generally comprises tip cutting surface 202 and bottom cutting surfaces 204. As shown, tip cutting surface 202 is arranged in a first plane that is 15° to 165° relative the cutting plane of bottom cutting surface 204. In a preferred embodiment, tip cutting surface is arranged in the first plane approximately 90° relative to the cutting plane of bottom cutting surface 204.

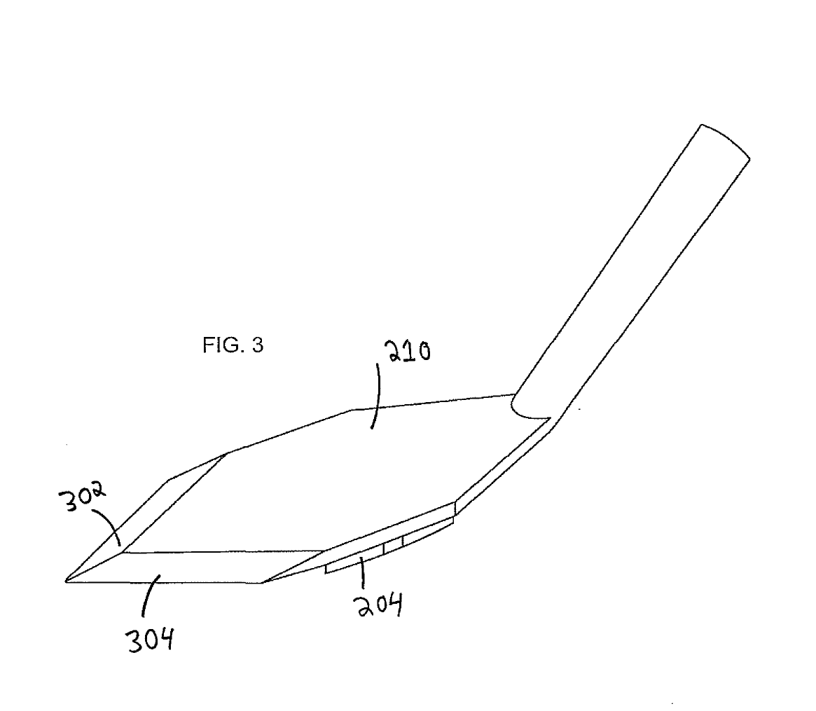

FIGS. 2-9 provide detailed views of tip cutting surface 202 and bottom cutting surface 204. The bottom of tip cutting surface 202 is formed from two intersecting surfaces 206 and 208 as depicted in FIG. 2. Similarly, the top of tip cutting surface 202 is formed from intersecting surfaces 302 and 304 as depicted in FIG. 3. Preferably, surfaces, 206, 208, 302, and 304 all have the same surface area and are angled away from base 210 at the same angle toward cutting tip 212. Preferably, the surfaces 206, 208, 302, and 304 are angled away from the base 210 toward angled tip 212 at an angle of 2° to 35°. In a preferred embodiment, the width of cutting surface 202 is approximately 0.5 mm-4 mm and the length of the cutting surface 202 is approximately 3 mm-20 mm. Base 210 preferably has a thickness of 40 μm-600 μm.

Bottom cutting surfaces 204 are formed extending downward from the sides of base 210 preferably just behind surfaces 206 and 208. Preferably, bottom cutting surfaces 204 have a height of approximately 10 to 300 μm as measured extending downward from base 210. Bottom cutting surfaces 204 also preferably have a length of up to 4.0 mm.

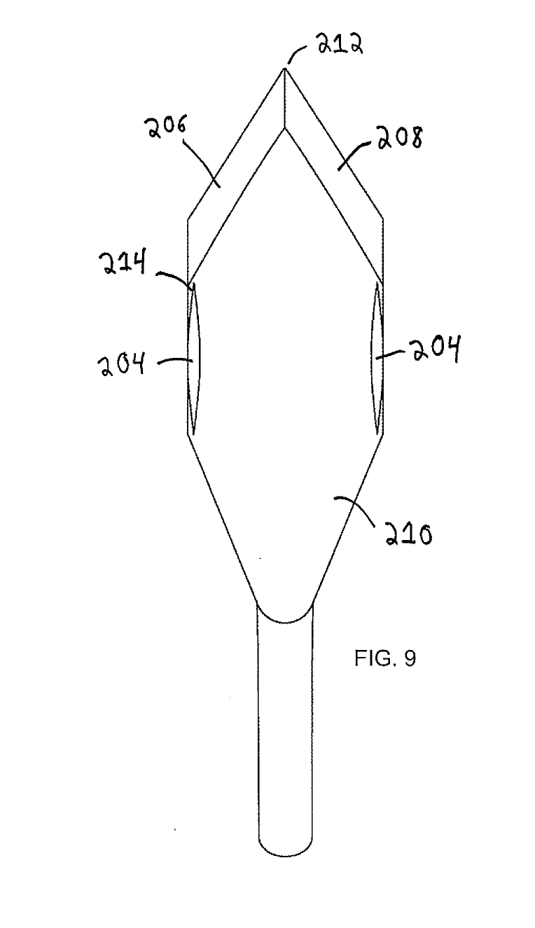

As best shown in FIGS. 2 and 9, each bottom cutting surface 204 has two angled planar surfaces which meet to form tip 214. The two angled planar surfaces generally meet at an angle of 2° to 35°. After tip 212 has been inserted to make an incision and the thickness of cutting tip 202 widens the tunnel incision into the corneal tissue, tip 214 on cutting surface 204 causes downward incision of the tunnel floor to be formed along the edges of the tunnel corneal. The advantage of the method and tool 100 to carry out self-sealing corneal and scleral valve incisions is first of all better tightness during surgery as well as in the postoperative period. Improved tightness of the wound during the surgery improves the stability of the anterior chamber or vitreous chamber and improves the safety of the treatment. In addition, IOP fluctuation is reduced while removing tools from the eye, which reduces the risk of some complications associated with the surgery. Improved tightness of the wound in the final phase of the procedure allows to abandon intracorneal fluid delivery in order to seal the wound or reduce the amount of fluid to be administered, which accelerates the healing process. Improved tightness of the wound reduces the risk of the most serious post-operative complication i.e.: endophthalmitis. Another advantage of the described tool is the reduced risk of unintentional extension of the tunnel laterally, e.g., when the patient moves the eye while the incision is being performed.

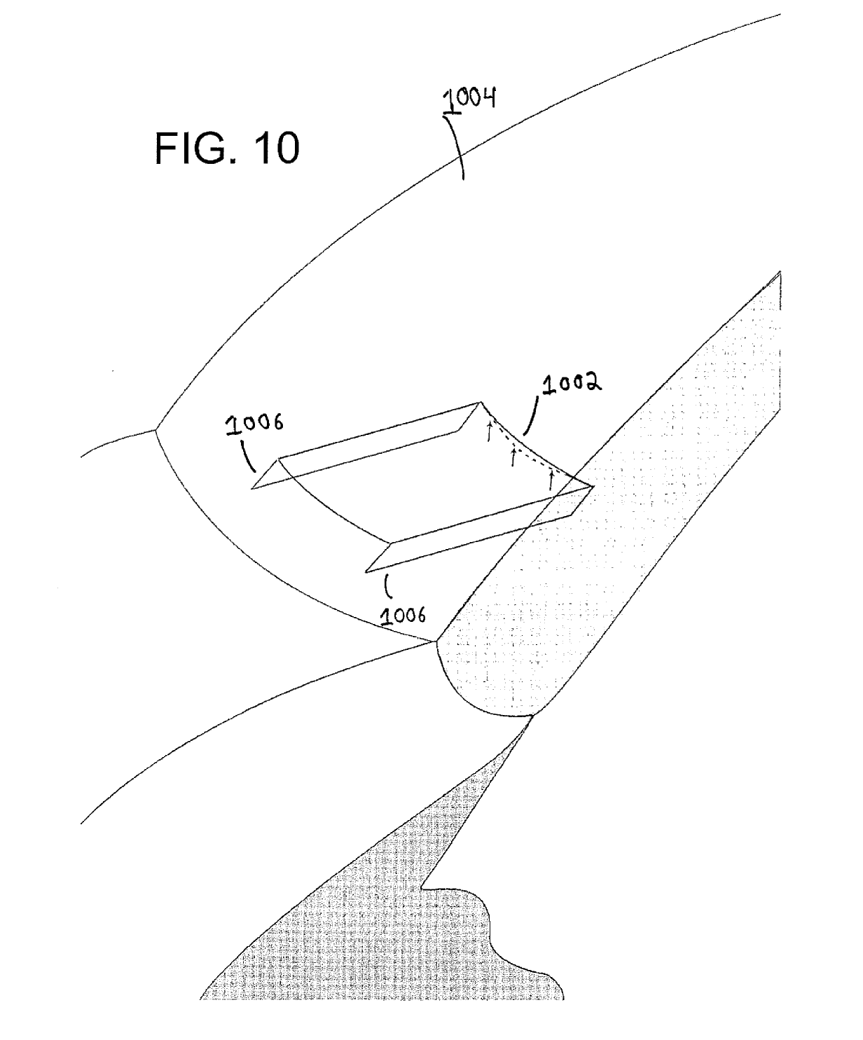

An example of a tunnel incision 1002 that was made in cornea 1004 using tool 100 is depicted in FIG. 10. Cutting tip 212 makes the initial incision into cornea and cutting tip 212 widens and deepens the tunnel incision 1002. As should be apparent, the width of tunnel incision 1002 is substantially the same as the width of base 210.

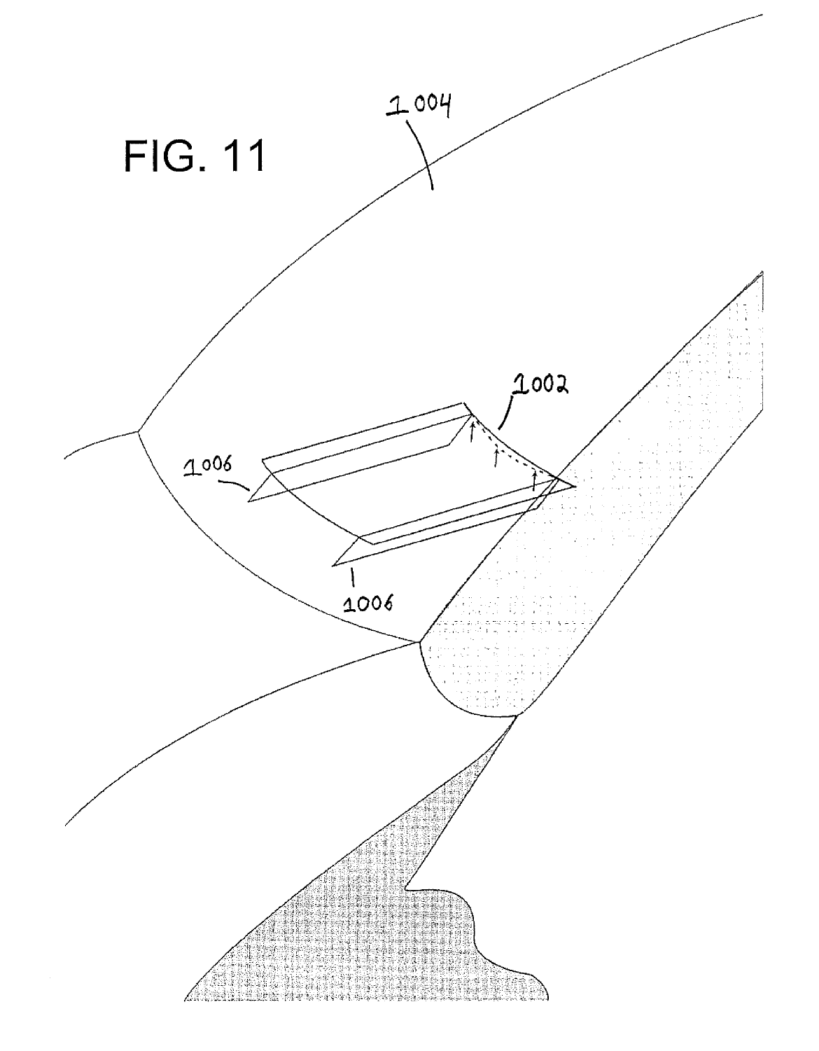

After the initial tunnel incision 1002 is made using cutting tip 202, cutting tip 214 on cutting surfaces 204 causes downward incision 1006 of the tunnel floor to be formed along the edges of the tunnel corneal incision 1002. Another example of a tunnel incision that can be made using tool 100 is depicted in FIG. 11. In this example of FIG. 11, the downward incision is formed near the edge of the tunnel corneal incision rather than at the edge.

Tunnel incisions of similar architecture can also be made by means of a femtosecond, nanosecond or another laser having the ability to perform cuts in the eye tissue.

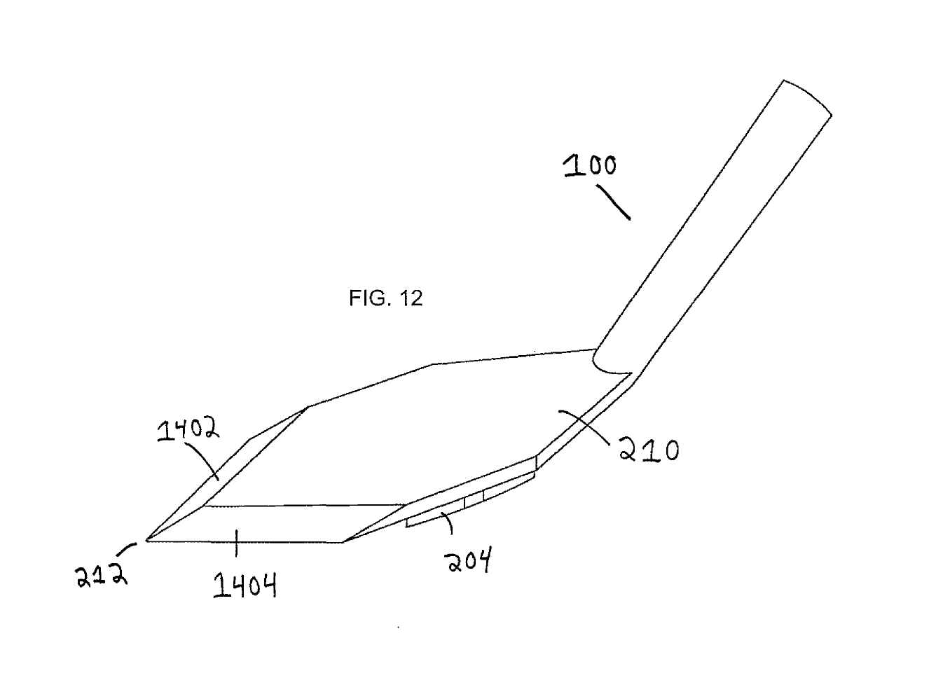

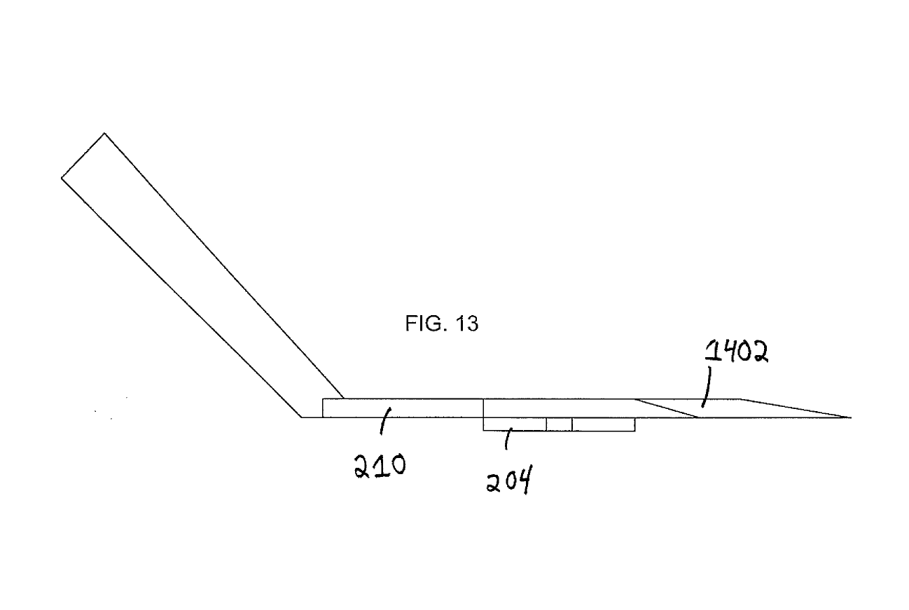

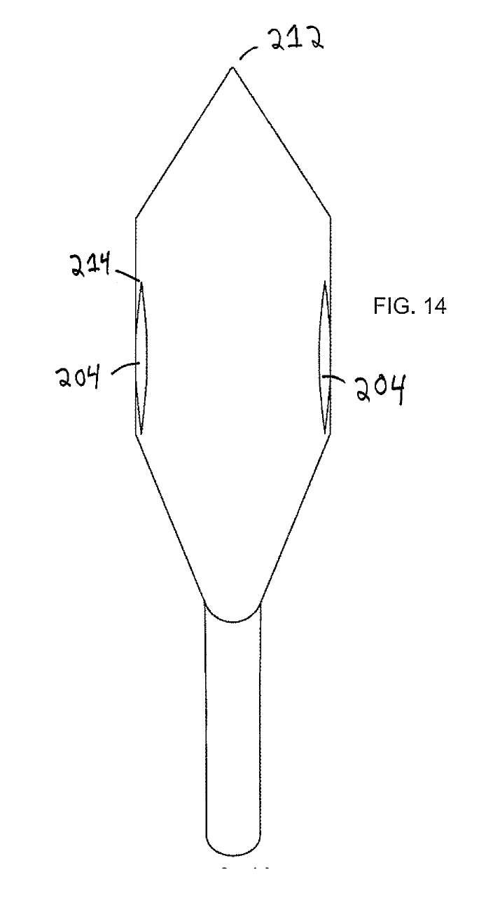

An alternate embodiment of tool 100 is depicted in FIGS. 12-14. Because the tools depicted in FIGS. 14-16 share much in common with the tool depicted in FIGS. 1-9, only the differences will be described for clarity. As shown, cutting tip 212 in this embodiment is formed by the meeting of beveled surfaces 1402 and 1404 and the bottom of base 210 (rather than the meeting of four beveled surfaces as in FIGS. 1-9). Otherwise, the relative dimensions of beveled surfaces 1402 and 1404 are relatively similar to that of surfaces 302 and 304.

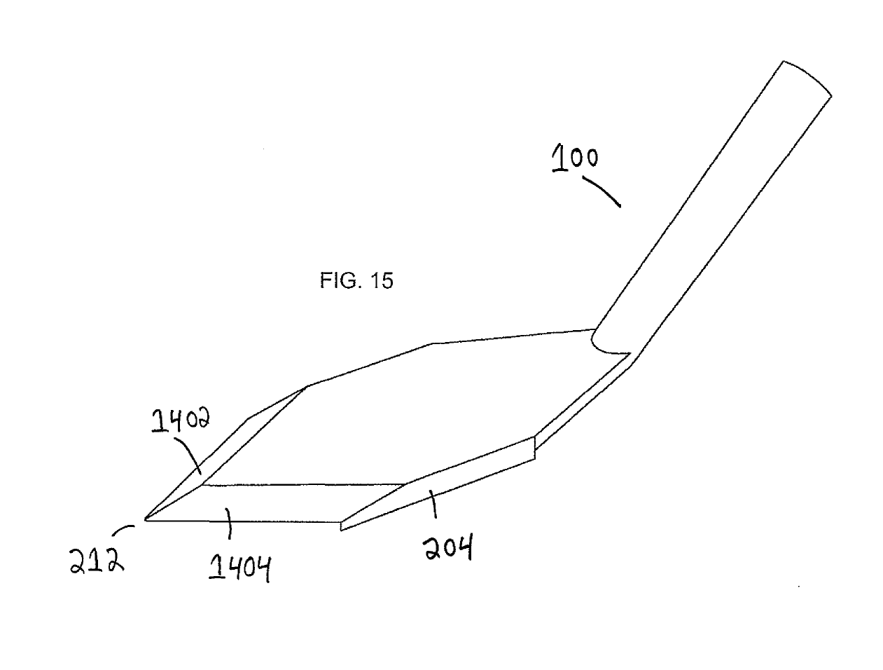

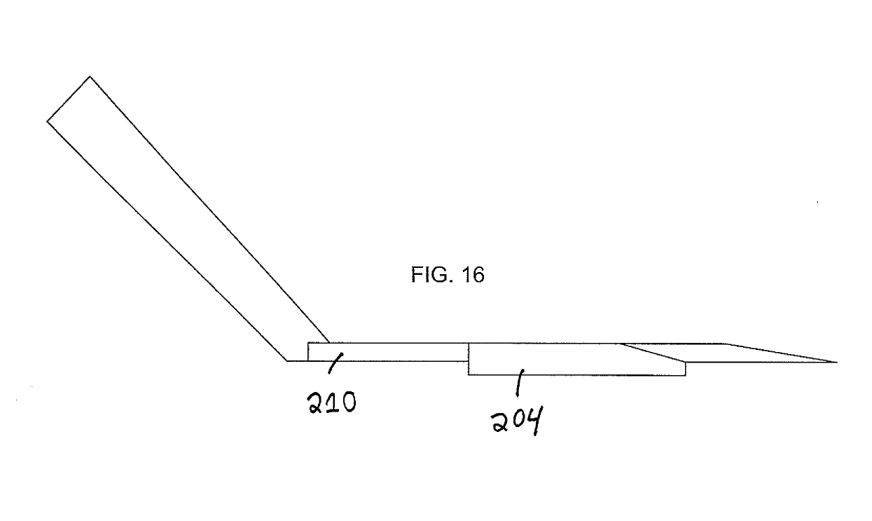

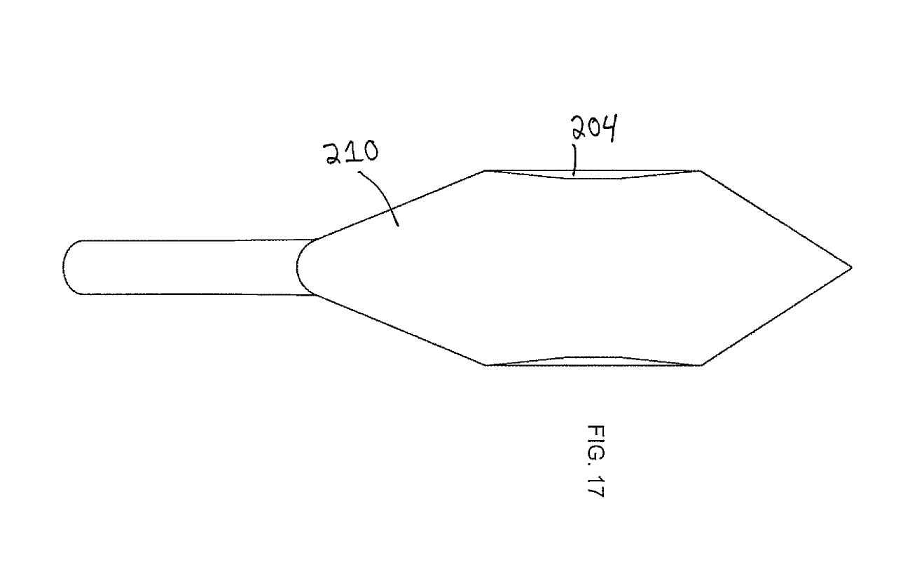

A second alternate embodiment of tool 100 is depicted in FIGS. 15-17. In this embodiment, cutting tip 212 is formed by the meeting of beveled surfaces 1402 and 1404 and the bottom of base 210 similar to that of the embodiment depicted in FIGS. 14-16. However, in the current embodiment, the design of cutting surfaces 204 has been modified. As best shown in FIG. 16, cutting surface 204 has been modified to extend down directly from the side of base 210 rather than from the bottom surfaces of base 210. As a result, cutting surfaces 204 are much thinner as depicted in FIG. 17. Also, since cutting surfaces 204 extend down from the side of base 210, they can extend forward all the way to meet the rear edges of beveled surfaces 1402 and 1404 as depicted in FIG. 15.

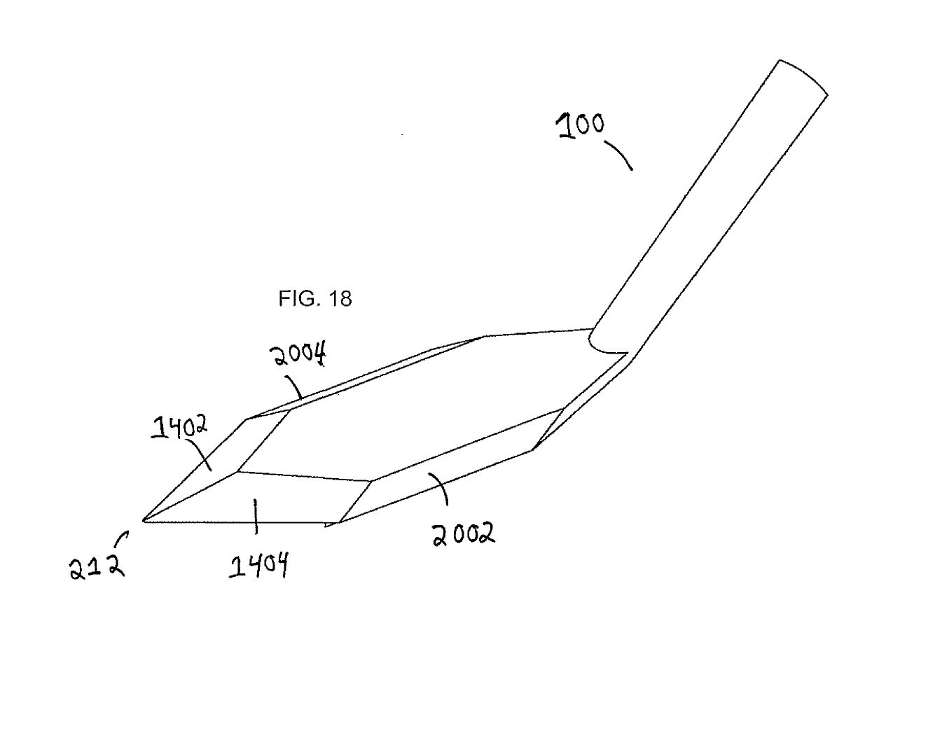

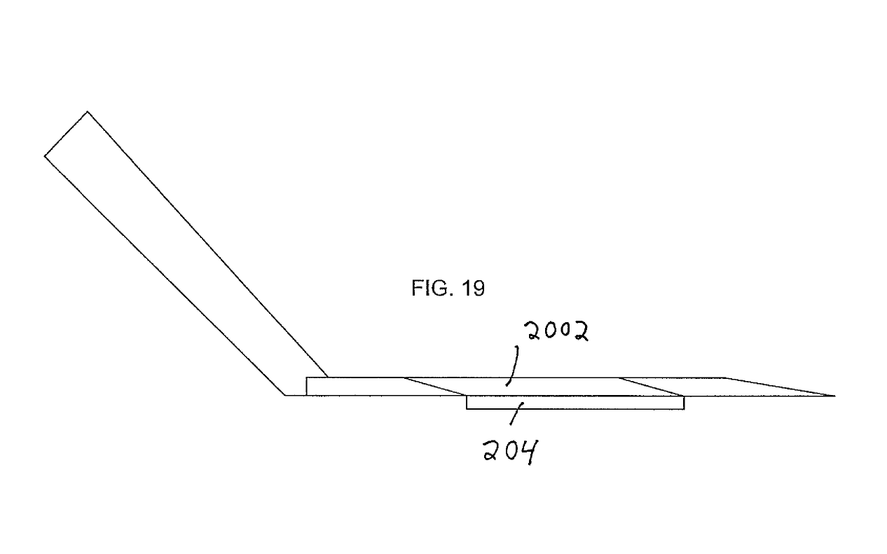

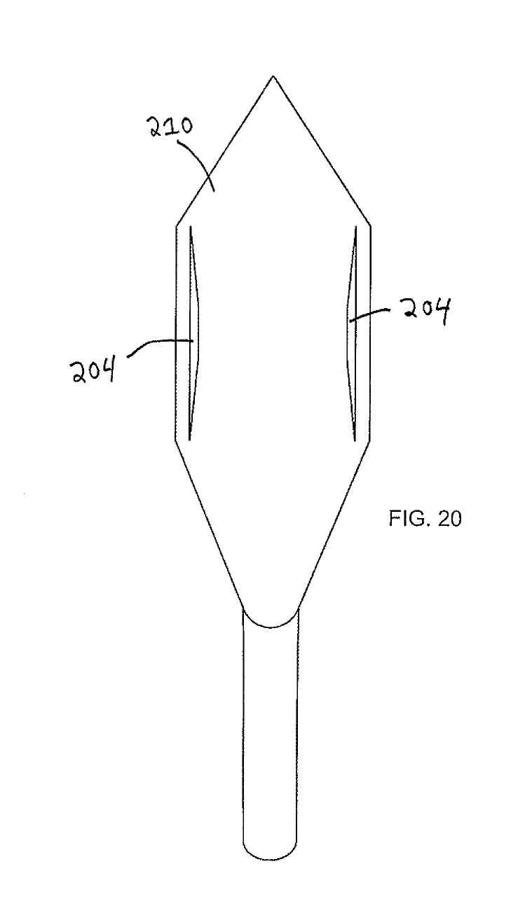

A third alternate embodiment of tool 100 is depicted in FIGS. 18-20. In this embodiment, the beveled surfaces 1402 and 1404 have been shortened and intersect with rear beveled surfaces 2002 and 2004. Cutting surfaces 204 preferably are located directly beneath rear beveled surfaces 2002 and 2004 as shown in FIG. 19. Further, as shown in FIG. 20, cutting surfaces are inset away from the edge of base 210 further than is depicted in the other embodiments disclosed here.

1. A method of performing a tunnel incision in a cornea or a sclera comprising:
making the tunnel incision into the cornea of a first thickness;
making two side incisions near the side edges of the tunnel incision,
wherein the two side incisions are performed at an angle intersecting a plane of the tunnel incision, and
wherein the two side incisions result in a partial downward incision of a tunnel floor of the tunnel incision on the side edges resulting in creation of a flap from inner layers of the cornea, the flap forming a valve that facilitates sealing of the tunnel incision and the two side incisions.

2. A tool for performing a tunnel incision in a cornea or a sclera comprising:
a body comprising a cutting tip for making the tunnel incision into the cornea of a first thickness;
two cutting planes extending downward from a bottom surface of the body near opposing sides of the body for performing two side incisions in the cornea near the tunnel incision,
wherein a tip of the two cutting planes is set at an angle of 60° to 120° relative to a plane of the base.

3. The tool according to claim 2, wherein the two cutting planes extending downward have a height from 40 to 160 μm as measured from the bottom surface of the body.

4. The method according to claim 1, wherein the tunnel incision is performed by a femtosecond laser or nanosecond laser.
